(19) United States
(12) Patent Application Publication
(10) Pub. No.: US 2006/0257482 A1
(43) Pub. Date: Nov. 16, 2006

(54) Modified release, multiple unit drug delivery systems

(76) Inventors: Patrik Kumar, Muzaffarpur Bihar (IN); Girish Kumar Jain, Pitampura (IN); Ashok Rampal, Amritsar (IN); Ravikumar Nithyanandam, Periyar (IN); Rajeev Singh Raghuvanshi, New Delhi (IN)

Correspondence Address: RANBAXY INC., 600 COLLEGE ROAD EAST, SUITE 2100, PRINCETON, NJ, 08540, US

(21) Appl. No.: 10/517,101

(22) PCT Filed: Jun. 9, 2003
(86) PCT No.: PCT/IB03/02186

§ 371 Date: Apr. 5, 2006

(30) Foreign Application Priority Data
Jun. 7, 2002 (IN) 617/DEL/2002
Nov. 15, 2002 (IN) 1157/DEL/2002
Mar. 6, 2003 (IN) 234/DEL/2003

(51) Int. Cl. A61K 31/553; A61K 31/554; A61K 31/522; A61K 31/496; A61K 31/366; A61K 31/22; A61K 31/137; A61K 9/26

(52) U.S. Cl. 424/469; 424/470; 514/211.07; 514/252.15; 514/423; 514/460; 514/548; 514/263.32; 514/649; 514/355; 514/253.08

(57) ABSTRACT

The invention relates to novel modified release multiple unit systems, and methods of preparing these systems, which can be easily compressed into tablets or filled into capsules or sachets without affecting the desired release characteristics of the pharmaceutical active ingredients incorporated within the systems. The multiple unit tablet includes multiple units. Each unit includes at least one core having an outer surface, a first coating layer surrounding at least a portion of the outer surface of the core and having an outer surface, one or more rate controlling polymers, and one or more one active pharmaceutical ingredients. The coating layer includes one or both of the one or more active pharmaceutical ingredients and the one or more rate controlling polymers. The tablet may further include an outer layer on the outer surface of the unit which includes a material that is one or both of elastic and compressible. The material may be a wax materials, such as polyethylene glycol's (PEGS).

### FIELD OF THE INVENTION

The technical field of the invention relates to modified release multiple unit systems, and methods of preparing these systems, which can be easily compressed into tablets or filled into capsules or sachets without affecting the desired release characteristics of the pharmaceutical active ingredients incorporated within the systems.

### BACKGROUND OF THE INVENTION

The need to improve the clinical results of modified release formulations is well documented in the prior art This is particularly important for drugs that have short half-lives, have region specific absorption, produce gastric irritation, or have other side effects at high plasma concentrations. One of the most common methods of achieving modified drug release involves the use of monolithic systems designed to have modified release characteristics. These monolithic systems vary from osmotic drug delivery systems to bioerodible or non-erodible matrix based systems.

Although a major portion of the modified release formulations currently prescribed are monolithic systems, they nonetheless suffer from a few serious drawbacks. Intentional or accidental breakdown of the delivery system is one of the limitations that may cause dose dumping. Dose dumping may lead to toxic or fatal effects, depending on the pharmaceutical compound. Further, the gastric emptying of the comparatively large monolithic systems is variable and is dependent on the presence or absence of food, as well as the type of food taken by the patient.

These disadvantages have prompted a shift in modified release technology from the use of monolithic systems to multiple unit systems, wherein each individual unit is formulated with modified release characteristics. The final dosage form consists of a collection of the multiple units, compressed into a tablet, or filled into a capsule or sachet. When administered, the individual units are dispersed freely into the gastrointestinal contents, avoiding the high local concentration of drug which may lead to irritation of gastrointestinal mucosa. Also, the performance of the dosage form is independent of inter- and intra-patient variability in gastric emptying time because of the small size of the individual units that make up the system. This technology has the added advantages of (1) allowing the production of numerous doses and strengths without the need for formulation or process changes; (2) delivery of incompatible agents together in a single dosage form; and (3) delivery of particles or individual units that have different release characteristics to achieve desired release profile.

Each individual unit of the multiple unit system is either: (a) an inert core or pellet coated with one or more layers of drug and other release controlling polymeric substances; or (b) a drug-containing core or pellet optionally coated with one or more layers of release controlling polymeric substances.

A common problem with modified release, multiple unit systems is the rupturing or cracking of the release controlling layers or membrane of the core, or the fragmentation of the core, due to the mechanical stress generated during the compression of cores or individual units into a tablet or filling into a capsule or sachet. Various approaches are described in the prior art for formulating multiple unit systems with a desired mechanical strength. For example, U.S. Pat. No. 4,713,248 discloses a water-based film comprising a homogenous combination of a water dispersible film forming agent and a polymeric substance that forms a film over a controlled release multiple unit formulation containing an active substance.

U.S. Pat. No. 5,783,215 describes the use of inert and non-soluble cores of glass or sand particles and soluble cores, such as sugar spheres, which are capable of withstanding mechanical stress, in combination with a plasticizing layer of a hydrophilic polymer containing the drug, optionally with additional layers of the polymer not containing the drug, layered between the core and the release controlling membrane.

### SUMMARY OF THE INVENTION

In one general aspect there is provided a multiple unit dosage form that includes multiple units. Each unit includes at least one core having an outer surface; a first coating layer surrounding at least a portion of the outer surface of the core and having an outer surface, the coating layer including one or both of one or more active pharmaceutical ingredients and one or more rate controlling polymers; and an outer layer. The outer layer includes a material that is one or both of elastic and compressible.

Embodiments of the multiple unit dosage form may include one or more of the following features. For example, the core may include the one or more rate controlling polymers. The core may include the one or more active pharmaceutical ingredients. The core may include the rate controlling polymer and the active pharmaceutical ingredient. The first coating layer may include the one or more active pharmaceutical ingredients.

The core may include one or more of sugar, a non-pareil seed, microcrystalline cellulose, celphere, sand silicon dioxide, glass, plastic, polystyrene, hydroxypropyl methylcellulose. The sugar may include one or more of glucose, mannitol, lactose, xylitol, dextrose, and sucrose. The core may include one or more of an insoluble material, a soluble material, and a swellable material.

The rate controlling polymer may include one or more of cellulosic polymers, methacrylic acid polymers, and waxes. The rate controlling polymer may include one or more of ethylcellulose, hydroxypropyl methylcellulose, hydroxypropyl cellulose, methylcellulose, carboxymethylcellulose, hydroxymethylcellulose, and hydroxyethylcellulose, hydroxypropylmethyl phthalate, cellulose acetate phthalate, and cellulose acetate trimellitate.

The one or more active pharmaceutical ingredients may include one or more of antidepressants, antidiabetics, antiulcers, analgesics, antihypertensives, antibiotics, antipsychotics, antineoplastics, antimuscarinics, diuretics, antimigraine agents, antivirals, anti-inflammatory agents, sedatives, antihistaminics, antiparasitic agents, antiepileptics and lipid lowering agents. The one or more active pharmaceutical ingredients may include one or more of enalapril, captopril, benazepril, lisinopril, ranitidine, famotidine, ranitidine bismuth citrate, diltiazem, propranolol, verapamil, nifedipine, acyclovir, ciprofloxacin, simvastatin, atorvastatin, lovastatin, venlafaxine, citalopram, paroxetine, selegiline, midazolam, fluoxetine, acarbose, buspirone, nimesulide, captopril nabumetone, glimepiride, glipizide, etodolac, nefazodone and their pharmaceutically acceptable salts. The one or more active pharmaceutical ingredients may be one or both of glipizide and venlafaxine or their salts.

The multiple unit dosage form may further include one or more additional layers. The additional layers are positioned between (a) one or more of the core and the first coating layer and (b) surrounding at least a portion of the first coating layer. The one or more additional layers include one or more of a seal coat, a film forming layer, a rate controlling polymer, and an active pharmaceutical ingredient. The seal coat may be one or more of hydroxypropyl methylcellulose, polyvinyl pyrrolidone, and methacrylic acid copolymers. The film forming layer may be one or more of ethyl cellulose, hydroxypropyl methylcellulose, hydroxypropyl cellulose, methyl cellulose, carboxymethylcellulose, hydroxymethylcellulose, hydroxyethylcellulose, hydroxypropyl methyl phthalate, cellulose acetate, cellulose acetate trimelliatate, cellulose acetate phthalate, waxes, polyethylene glycol, and methacrylic acid polymers.

The multiple unit dosage form may further include an outer layer on the outer surface of the unit and the outer surface includes a material that is one or both of elastic and compressible. The material in the outer layer may be one or more wax materials. The wax material may be one or more polyethylene glycols (PEGs). The PEGs may differ by molecular weight. The polyethylene glycol (PEG) may be one or more of PEG 600, PEG 4000, PEG 6000, PEG 8000, and PEG 20000. The waxy material may be from about 1% to about 15% by weight of the total tablet weight or from about 1% to about 100% by weight of the weight of the core and first coating layer. The waxy material may be applied to each unit as a solution, suspension, dispersion, or hot melt technique. The solution, suspension, or dispersion may be made using a solvent. The solvent may be one or more of methylene chloride, isopropyl alcohol, acetone, methanol, ethanol, and water.

The active pharmaceutical ingredient may be glipizide and may be present in one or both of the core and the first coating layer. The multiple unit dosage form may further include a buffering agent with the glipizide in one or both of the core and the first coating layer. The buffering agent may be one or more of dibasic sodium phosphate, sodium ascorbate, meglumine, sodium citrate trimethanolamine, sodium hydroxide, potassium hydroxide, calcium hydroxide, magnesium hydroxide, ammonia, tertiary sodium phosphate, diethanolamine, ethylenediamine, and L-lysine.

In the multiple unit dosage form, one or more of the core and the first coating layer may include one or more pharmaceutically acceptable excipients. The pharmaceutically acceptable excipients may include surfactants, binders, diluents, disintegrants, lubricants, glidants, plasticizers, stabilizers, and coloring agents. The surfactants may include one or more of a non-ionic surfactant, an ionic surfactant, mono fatty acid esters of polyoxyethylene sorbitan, polyoxyethylene (20) sorbitan monooleate (Tween 80), polyoxyethylene (20) sorbitan monostearate (Tween 60), polyoxyethylene (20) sorbitan monolaurate (Tween 20), an anionic surfactant, sodium lauryl sulfate, polyoxyethylene castor oil derivative, polyoxyethyleneglycerol triiricinoleate castor oil, polyoxyl 35 castor oil, Cremophor EL, and Vitamin E TPGS, d-alpha-tocopheryl polyethylene glycol 1000 succinate, polyethoxylated fatty acids and their derivatives, polyethylene glycol 400 distearate, polyethylene glycol-20 dioleate, polyethylene glycol 4-150 mono dilaurate, polyethylene glycol-20 glyceryl stearate, alcohol-oil transesterification products, polyethylene glycol-6 corn oil, polyglycerized fatty acids, polyglyceryl-6 pentaoleate, propylene glycol fatty acid esters, propylene glycol monocaprylate, mono and diglycerides, glyceryl ricinoleate, sterol and sterol derivatives, sorbitan fatty acid esters and their derivatives, polyethylene glycol-20 sorbitan monooleate and sorbitan monolaurate, polyethylene glycol alkyl ether or phenols, polyethylene glycol-20 cetyl ether, polyethylene glycol-10-100 nonyl phenol, sugar esters, sucrose monopalmitate, polyoxyethylene-polyoxypropylene block copolymers, poloxamer, sodium caproate, sodium glycocholate, soy lecithin, sodium stearyl fumarate, propylene glycol alginate, octyl sulfosuccinate disodium, and palmitoyl carnitine.

The binders may include one or more of methyl cellulose, hydroxypropyl cellulose, hydroxypropyl methylcellulose, polyvinylpyrrolidone, gelatin, gum arabic, ethyl cellulose, polyvinyl alcohol, pullulan, pregelatinized starch, agar, tragacanth, sodium alginate, and propylene glycol. The diluents may include one or more of calcium carbonate, calcium phosphate-dibasic, calcium phosphate-tribasic, calcium sulfate, microcrystalline cellulose, silicified microcrystalline cellulose, cellulose powdered, dextrates, dextrins, dextrose excipients, fructose, kaolin, lactitol, lactose, mannitol, sorbitol, starch, starch pregelatinized, sucrose, sugar compressible, and sugar confectioners. The disintegrants include one or more of starch, croscarmellose, crospovidone, and sodium starch glycolate. The lubricants and glidants include one or more of colloidal anhydrous silica, stearic acid, magnesium stearate, calcium stearate, talc, hydrogenated caster oil, sucrose esters of fatty acid, microcrystalline wax, yellow beeswax, and white beeswax. The plasticizers include one or more of polyethylene glycol, triethyl citrate, triacetin, diethyl phthalate, and dibutyl sebacate. The stabilizers include one or more of antioxidants, buffers, and acids.

The multiple unit dosage form may further include one or more pharmaceutically acceptable excipients around the individual units. The dosage form may be a tablet and the tablet may be formed by application of a compressive force. The dosage form may be a capsule.

The active pharmaceutical ingredients of the multiple unit dosage form may be one or more of atorvastatin and amlodipine, metformin and glipizide, simvastatin and ramipril, simvastatin and amlodipine, metformin XL and glipizide XI, ramipril and atorvastatin, ramipril and amlodipine, metformin XL and glimiperide, fosinopril and amlodipine.

In another general aspect, there is provided a process for the preparation of a multiple unit dosage form. The process includes providing at least one core having an outer surface, forming a coated core by applying one or more coating layers to the core such that the one or more coating layers surround at least a portion of the outer surface of the core or the coating layers, forming an individual unit by applying a waxy material to the coated core to form a wax layer, and combining one or more units to form a multiple unit dosage form. One or both of the core and the coating layers includes one or more rate controlling polymers and active pharmaceutical ingredients.

Embodiments of the process may include one or more of the following features. For example, the process may further include applying one or both of a seal layer or a film forming layer between the core and the coating layer, between the one or more coating layers, and between the one or more coating layers and the wax layer. The waxy material may be one or more polyethylene glycols (PEGs) of one or more molecular weights. The polyethylene glycols (PEG) may be one or more of PEG 600, PEG 4000, PEG 6000, PEG 8000, and PEG 20000. The waxy material may be from about 1% to about 15% by weight of the total tablet weight. The waxy material may be from about 1% to about 100% by weight of the weight of the core and the one or more coating layers.

Applying the waxy material may include applying a coating of a solid waxy material by using a hot melt technique. Applying the waxy material may include applying a coating of waxy material by using as one or more of a solution, a suspension, and a dispersion. The solution or the suspension may be prepared in a solvent. The solvent may be selected from one or more of methylene chloride, isopropyl alcohol, acetone, methanol, ethanol, and water.

The core may be an inert core. The core may include one or more pharmaceutically acceptable excipients. The core may include one or more active pharmaceutical ingredients. The one or more active pharmaceutical ingredients may be one or more of antidepressants, antidiabetics, antiulcers, analgesics, antihypertensives, antibiotics, antipsychotics, antineoplastics, antimuscarinics, diuretics, antimigraine agents, antivirals, anti-inflammatory agents, sedatives, antihistaminics, antiparasitic agents, antiepileptics and lipid lowering agents. The one or more active pharmaceutical ingredients may be one or more of enalapril, captopril, benazepril, lisinopril, ranitidine, famotidine, ranitidine bismuth citrate, diltiazem, propranolol, verapamil, nifedipine, acyclovir, ciprofloxacin, simvastatin, atorvastatin, lovastatin, venlafaxine, citalopram, paroxetine, selegiline, midazolam, fluoxetine, acarbose, buspirone, nimesulide, captopril, nabumetone, glimepiride, glipizide, etodolac, nefazodone and their pharmaceutically acceptable salts. In particular, the active pharmaceutical ingredient may be venlafaxine or glipizide.

The core may be prepared by extrusion-spheronization. The extrusion-spheronization process may include granulating an inert core material with or without other pharmaceutical excipients with a binder solution to form a wet mass, passing the wet mass through an extruder to form extrudates, and spheronizing the extrudates. The core may be prepared by granulation. The granulation process may include wetting a dry mix of core material with or without other pharmaceutical excipients with a binder solution.

The units may be prepared by coating the cores with active pharmaceutical ingredients and rate controlling polymers. The units may be prepared by coating cores with a first layer comprising an active pharmaceutical ingredient and a second outer layer comprising a rate controlling polymer.

The process may further include applying a seal coat or a film forming layer between the core and the subsequent layers. The process may further include applying a seal coat or a film forming layer between a layer comprising an active pharmaceutical ingredient and a layer comprising a release rate controlling polymer

The rate controlling polymer may include one or more of cellulosic polymers, methacrylic acid polymers, and waxes. The rate controlling polymer may be one or more of ethylcellulose, hydroxypropyl methylcellulose, hydroxypropyl cellulose, methylcellulose, carboxymethylcellulose, hydroxymethylcellulose, hydroxyethylcellulose, hydroxypropylmethyl phthalate, cellulose acetate phthalate, and cellulose acetate trimellitate.

In another general aspect, a method for preparing a modified release multiple unit dosage form includes providing a core having a coating, forming individual units by coating the coated core with a coating material that is one or both of compressible and elastic, and forming the dosage form by combining one or more individual units. One or both of the core and the coating may be one or more rate controlling polymers and one or more active pharmaceutical ingredients.

Embodiments of the method of preparing a modified release multiple unit dosage form may include one or more of the following features, including any one or more of the features described above. For example, the coating material may be a waxy material. The coating material may be a polyethylene glycol. Combining one or more individual units may include filling the individual units into a capsule or sachet or compressing the individual units into a tablet.

In another general aspect, a method of treating a medical condition includes administering a multiple unit tablet for oral ingestion. Each unit includes a core, one or more layers surrounding the core, and an outer layer. The core includes one or more of a pharmaceutically acceptable excipient, an active pharmaceutical ingredient, and a rate controlling polymer. The one or more layers includes one or more of a pharmaceutically acceptable excipient, an active pharmaceutical ingredient, a rate controlling polymer, a sealing layer, and a film forming layer. The outer layer includes a material that is one or both of compressible or elastic to partially or completely absorb a compressive force exerted in combining the units.

Embodiments of the method of treating a medical condition may include one or more of the following features, including any one or more of the features described above. For example, the material of the outer layer may be a waxy material. The waxy material may be one or more polyethylene glycols of different molecular weights.

In another general aspect, a combination drug, multiple unit dosage form includes first units and second units. Each first unit includes at least one core having an outer surface, a first coating layer surrounding at least a portion of the outer surface of the core and having an outer surface, and an outer layer surrounding at least a portion of an outer surface of the first coating layer, the first coating layer including a first active pharmaceutical ingredient. Each second unit includes at least one core having an outer surface, a first coating layer surrounding at least a portion of the outer surface of the core and having an outer surface, and an outer layer surrounding at least a portion of an outer surface of the first coating layer, the first coating layer including a second active pharmaceutical ingredient. One or both of the cores and the coating layers may include the rate controlling polymer. One or both of the outer layers may include a waxy material.

Embodiments of the combination drug, multiple unit dosage form may include one or more of the following features, including any one or more of the features described above. For example, waxy material may include one or more polyethylene glycols.

In another general aspect, a multiple unit dosage form includes multiple units. Each unit includes at least one core having an outer surface and comprising one or more one active pharmaceutical ingredients; and a coating layer surrounding at least a portion of the outer surface of the core, having an outer surface and comprising a waxy material.

Embodiments of the dosage form may include one or more of the following features. For example, the waxy material may be one or more polyethylene glycols of different molecular weights. The dosage form may be a tablet or a capsule.

In another general aspect, a multiple unit dosage form includes multiple units. Each unit includes at least one core having an outer surface and a first coating layer surrounding at least a portion of the outer surface of the core and having an outer surface. The coating layer includes glipizide or its pharmaceutically acceptable salt and optionally one or more rate controlling polymers.

In one embodiment, the pharmaceutically acceptable salt comprises one or more of mineral acid salts, organic acid salts, and organosulphonic acid salts.

In another general aspect, a modified release multiple unit system includes units of glipizide. The units include an inert core; a drug layer surrounding the inert core, the drug layer including glipizide; and a rate controlling polymer layer surrounding the drug layer.

Embodiments of the modified release multiple unit system may include one or more of the following features. For example, the system may be a tablet or a capsule.

In another general aspect, a modified release multiple unit system includes units of glipizide. The units include an inert core; a drug layer surrounding the inert core; a rate controlling polymer layer surrounding the drug layer; and a waxy layer surrounding the drug layer.

Embodiments of the modified release multiple unit system may include one or more of the following features. For example, the system may be a tablet or a capsule. The units can be compressed into tablet, or filled into a capsule or a sachet; without affecting the desired release characteristics of drug.

In another general aspect, a modified release multiple unit system includes units of venlafaxine. The units include an inert core; a drug layer surrounding the inert core; and a rate controlling polymer layer surrounding the drug layer.

Embodiments of the modified release multiple unit system may include one or more of the following features. For example, the system may be a tablet. The units can be compressed into tablet without affecting the desired release characteristics of drug.

In another general aspect, a modified release multiple unit system includes units of venlafaxine. The units include an inert core; a drug layer surrounding the inert core; a rate controlling polymer layer surrounding the drug layer; and a waxy layer surrounding the rate controlling polymer layer.

Embodiments of the modified release multiple unit system may include one or more of the following features. For example, the system may be a tablet. The units can be compressed into tablet without affecting the desired release characteristics of the venlafaxine.

In another general aspect, a modified release multiple unit system comprises units of a drug. The units include an inert core; a drug layer surrounding the inert core; a rate controlling polymer layer surrounding the drug layer, and a waxy layer surrounding the rate controlling polymer layer.

Embodiments of the modified release multiple unit system may include one or more of the following features. For example, the system may be compressed into tablet, or filled in capsule or sachet without affecting the desired release characteristics of drug.

In another general aspect, a process for the preparation of a modified release multiple unit system of a drug includes the steps of coating inert pellets with a drug and rate controlling polymer layer; coating with a waxy layer; optionally blending with pharmaceutically acceptable excipients; compressing into a tablet, or filing into a capsule or a sachet of suitable size.

In another general aspect, a process for the preparation of a modified release multiple unit system of drug includes the steps of coating inert pellets with a drug and rate controlling polymer layer; coating with a waxy layer; optionally blending with pharmaceutically acceptable excipients; and compressing into tablet of suitable size.

Embodiments of the modified release multiple unit system may include one or more of the following features. For example, the drug may be venlafaxine or a pharmaceutically acceptable salt.

In another general aspect, a process for the preparation of modified release multiple unit system of drug includes the steps of coating drug containing cores with a rate controlling polymer layer; coating the rate controlling polymer layer with a waxy layer; optionally blending with pharmaceutically acceptable excipients; and compressing into a tablet, or filling into a capsule or a sachet of suitable size.

The details of one or more embodiments of the inventions are set forth in the description below. Other features, objects, and advantages of the invention will be apparent from the description and claims.

### DETAILED DESCRIPTION OF THE INVENTION

As described above with respect to the difficulties associated with prior art compositions, there exists a need for universally applicable, multiple unit dosage form or systems of desired mechanical strength. The difficulties in the prior art are believed to be addressed by the techniques, compositions, and concepts described herein for a modified release, multiple unit system that can be easily compressed into a tablet or filled into a capsule or sachet without affecting the desired release characteristics of the drug. To address the above described problems of the prior art associated with mechanical stress due to compression or filling, the inventors have found that there are benefits to providing an outermost coating of a waxy material to each unit of the multiple unit systems. The inventors have found that the application of a coating of waxy material to each unit provides favorable mechanical properties that withstand cracking. Specifically, the coating of waxy material withstands cracking of the release controlling membrane when exposed to mechanical stress, for example, during compression into a tablet or filling into a capsule or sachet.

The inventors have applied the multiple unit dosage form or system techniques, compositions, and concepts to active pharmaceutical ingredients, including venlafaxine and glipizide. In so doing, the inventors have developed separate multiple unit dosage form or systems of venlafaxine and glipizide that are in the form of controlled release tablets in which the waxy layer is an optional component. These venlafaxine and glipizide controlled release, multiple unit tablets that include coated pellets of venlafaxine or glipizide, respectively, overcome the known problem of limited dosing associated with capsules. The term “controlled release” as used herein includes any type of modified release such as prolonged release, delayed release, sustained release, extended release and the like.

The waxy coating imparts a certain degree of elasticity or compressibility to the units and makes possible the compression of the multiple units into tablets or filling into capsules or sachets without altering the dissolution profile and hence the bioavailability and desired clinical effects. Further, this approach can be used over any types of pre-functional layers and irrespective of drug characteristics. Hence, the waxy coating provides a method for the preparation of modified or controlled release, multiple unit dosage forms or systems that include a final or outer coating of a waxy material and these units can be easily compressed into tablets, or filled into capsules or sachets without affecting the desired release characteristics of drug (e.g., dissolution profile, bioavailability, and clinical effects). In particular, the waxy layer can protect the release control polymer layer from cracking during compression, for example, during the production of tablets.

In general, the multiple units can be for use in any dosage forms, such as a tablet, capsule or sachet, and include a core or pellet, one or more layers around the pellet, and an outer waxy layer. The core or pellet can be entirely or partially an active pharmaceutical ingredient or an inert material, or a combination of both. The layers around the core may include one or more release or rate controlling polymers and/or active pharmaceutical ingredients. The layers also may be in the form of sealing or film forming layers around or between the polymer and active pharmaceutical ingredients. The various layers and core may optionally contain pharmaceutically acceptable excipients. The outer waxy layer may consist entirely of a waxy material or may be a mixture of a waxy material and one or more pharmaceutically acceptable functional excipients.

The multiple units of the improved multiple unit systems may contain (1) inert pellets or cores or (2) drug containing pellets or cores in which the drug is incorporated within the pellets or cores. Cores and pellets generally are used interchangeably herein. The inert core of the improved multiple unit systems is either a commercially available product or prepared in the laboratory. The inert core may be of any geometric shape, although spherical beads have the advantage of providing ease of uniform coating. The bead diameter may vary from about 50 μm to 700 μm. The pellet weight may vary from about 3% to about 40% by weight of the total tablet weight.

The commercially available inert cores include sugar spheres, non pariel seeds, celpheres and the like. The laboratory or otherwise manufactured cores may be prepared according to any suitable method including:
a. Extrusion-Spheronization: The inert core material with or without drug and other pharmaceutical excipients is granulated by addition of a binder solution. The wet mass is passed through an extruder equipped with a screen. The extrudates are spheronized in a marumerizer. The resulting spheroids or pellets are dried and sieved for further applications.
b. Granulation: The inert core material with or without drug and other pharmaceutical excipients is dry-mixed and then the mixture is wetted by addition of a binder solution in a high shear-granulator/mixer. The granules are kneaded after wetting by the combined actions of mixing and milling. The resulting granules or pellets are dried and sieved for further applications.

The material from which the inert pellet or core is prepared may be selected from one or more of pharmaceutically inert insoluble, soluble, and/or swellable materials, with or without pharmaceutically acceptable excipients. The insoluble inert core material may be, for example, one or more of sand (silicon dioxide), glass, microcrystalline cellulose (e.g., celpheres) or plastic (e.g., polystyrene) material. The soluble inert core material may be, for example, one or more sugar such as glucose, mannitol, lactose, xylitol, dextrose, sucrose, and the like. The swellable inert core material may be, for example, hydroxypropyl methylcellulose or a similar material. The core also can be a combination of two or more of these three general types of core materials.

Alternatively, drug-containing cores can also be prepared by completely or partially replacing the inert core material with one or more active pharmaceutical ingredients in the above two methods of preparing inert cores.

The improved, modified release multiple units may be prepared from inert cores by (a) coating the inert core with one or more drug and rate controlling polymer layers; or (b) coating the inert core with one or more drug layers and rate controlling polymer layers separately. Both of these options may contain a seal or film coat between the inert core and the drug layer and/or between the drug layer and the rate controlling polymer layer.

The improved, modified release multiple units also may be prepared from drug containing cores by (a) coating drug containing cores with rate controlling polymer; or (b) coating drug containing cores with drug and rate controlling polymer. Both of these options may contain a seal or film coat between the drug containing core and the polymer layer and/or over the polymer layer. The seal or film coat layer also can be formed between the drug containing core and the drug/polymer layer and/or over the drug/polymer layer.

The improved, modified release units are further processed by applying a final layer of a waxy material over each unit prepared by the above processes. Although the application of this waxy layer is the general rule, the inventors nonetheless have successfully formed tables from multiple units without the waxy layer. This may be dependent on, for example, the active pharmaceutical ingredient of the tablet.

The modified release units prepared by any of the above methods can be mixed with other pharmaceutically acceptable excipients, to the extent required or desired, and compressed into tablets or filled into capsules and sachets using techniques known in the art for these purposes. The final tablets or capsules may optionally be coated, if desired.

The drug layer of the improved multiple unit tablet includes one or more active pharmaceutical ingredients, and optionally includes other pharmaceutically acceptable excipients. The drug layer may be applied as an aqueous or non-aqueous solution or dispersion of drug in water or organic solvent, or mixtures thereof. The one or more drugs may be selected from, for example, one or more of antidepressants, antidiabetics, antiulcers, analgesics, antihypertensives, antibiotics, antipsychotics, antineoplastics, antimuscarinics, diuretics, antimigraine agents, antivirals, anti-inflammatory agents, sedatives, antihistaminics, antiparasitic agents, antiepileptics and lipid lowering agents.

Illustrative examples of drugs of the above classes include enalapril, captopril, benazepril, lisinopril, ranitidine, famotidine, ranitidine bismuth citrate, diltiazem, propranolol, verapamil, nifedipine, acyclovir, ciprofloxacin, simvastatin, atorvastatin, lovastatin, venlafaxine, citalopram, paroxetine, selegiline, midazolam, fluoxetine, acarbose, buspirone, nimesulide, captopril, nabumetone, glimepiride, glipizide, etodolac, nefazodone and their pharmaceutically acceptable salts.

The rate controlling polymer layer includes one or more polymers with or without other pharmaceutically acceptable excipients. This layer may be applied as an aqueous or non-aqueous solution or dispersion of polymers in a water or organic solvent. Suitable rate controlling polymers include one or more of cellulosic polymers such as ethylcellulose, hydroxypropyl methylcellulose, hydroxypropyl cellulose, methylcellulose, carboxymethylcellulose, hydroxymethylcellulose, and hydroxyethylcellulose; waxes; hydroxypropylmethyl phthalate; cellulose acetate phthalate; cellulose acetate trimellitate; and methacrylic acid polymers such as Eudragit® RL and RS. The single drug and rate controlling layer may contain the above described drug and polymers in the same layer. Based on the desired release profile, the controlled release polymer layer weight may constitute from about 5% to about 75% of the total tablet weight.

The waxy material may be selected from, for example, a range of polyethylene glycols (PEGs) of various molecular weights, such as PEG 600, PEG 4000, PEG 6000, PEG 8000, PEG 20000 and the like. In general, the waxy material should be at least of approximately as compressible or elastic as PEG. The waxy material lays may constitute, for example, from about 1% to about 15% by weight of the total tablet weight, although the amount may be varied up or down if necessary. The amount of the waxy material may vary from about 1% to about 100% by weight of the weight of the core and coating layer or one or more coating layers. The waxy layer is applied as a solution or suspension using any conventional coating technique known in the art, including spray coating in a conventional coating pan or fluidized bed processor, dip coating of each unit of a multiple unit system, or using a hot melt technique.

The solvents used for making a solution, dispersion, or suspension of the waxy material may be selected from, for example, one or more of methylene chloride, isopropyl alcohol, acetone, methanol, ethanol, and water. In general, the solvent should adequately dissolve, disperse, or suspend whichever waxy material or materials is selected.

The seal coat may include suitable polymers, such as hydroxypropyl methylcellulose, polyvinyl pyrrolidone, methacrylic acid copolymers and the like. The film forming coat or agents may include one or more of ethyl cellulose, hydroxypropyl methylcellulose, hydroxypropyl cellulose, methyl cellulose, carboxymethylcellulose, hydroxymethylcellulose, hydroxyethylcellulose, hydroxypropyl methyl phthalate, cellulose acetate, cellulose acetate trimelliatate, cellulose acetate phthalate, waxes such as polyethylene glycol, and methacrylic acid polymers such as Eudragit® RL and RS. Alternatively, the film forming layer or agents may be commercially available coating compositions including film-forming polymers marketed under various trade names, such as Opadry®. Film forming layers generally are provided for achieving a smooth surface and better appearance. Seal layer generally are applied to separate two incompatible layers, provide protection from moisture, etc. In general, the film forming layers and the seal layers may be the same or similar polymers used in different combinations or concentrations.

The other pharmaceutically acceptable excipients as used herein include surfactants, binders, diluents, disintegrants, lubricants, glidants, plasticizers, stabilizers and coloring agents.

Suitable surfactants include one or more of non-ionic and ionic (i.e., cationic, anionic and Zwitterionic) surfactants suitable for use in pharmaceutical compositions. For example, suitable surfactants include non-ionic surfactants such as mono fatty acid esters of polyoxyethylene sorbitan (e.g., polyoxyethylene (20) sorbitan monooleate (Tween 80), polyoxyethylene (20) sorbitan monostearate (Tween 60), polyoxyethylene (20) sorbitan monolaurate (Tween 20)); anionic surfactants (e.g., sodium lauryl sulfate); polyoxyethylene castor oil derivatives (e.g., polyoxyethyleneglycerol triiricinoleate or polyoxyl 35 castor oil (Cremophor EL)); and Vitamin E TPGS (d-alpha-tocopheryl polyethylene glycol 1000 succinate). Other suitable surfactants include polyethoxylated fatty acids and their derivatives (e.g., polyethylene glycol 400 distearate, polyethylene glycol-20 dioleate, polyethylene glycol 4-150 mono dilaurate, and polyethylene glycol-20 glyceryl stearate); alcohol-oil transesterification products (e.g., polyethylene glycol-6 corn oil); polyglycerized fatty acids (e.g., polyglyceryl-6 pentaoleate); propylene glycol fatty acid esters (e.g., propylene glycol monocaprylate); mono and diglycerides (e.g., glyceryl ricinoleate); sterol and sterol derivatives; sorbitan fatty acid esters and their derivatives (e.g., polyethylene glycol-20 sorbitan monooleate and sorbitan monolaurate); polyethylene glycol alkyl ether or phenols (e.g., polyethylene glycol-20 cetyl ether, polyethylene glycol-10-100 nonyl phenol); sugar esters (e.g., sucrose monopalmitate; polyoxyethylene-polyoxypropylene block copolymers known as “poloxamer”); and ionic surfactants (e.g., sodium caproate, sodium glycocholate, soy lecithin, sodium stearyl fumarate, propylene glycol alginate, octyl sulfosuccinate disodium, and palmitoyl carnitine).

Suitable binders include one or more of methyl cellulose, hydroxypropyl cellulose, hydroxypropyl methylcellulose, polyvinylpyrrolidone, gelatin, gum arabic, ethyl cellulose, polyvinyl alcohol, pullulan, pregelatinized starch, agar, tragacanth, sodium alginate, propylene glycol, and the like.

Suitable diluents include one or more of calcium carbonate, calcium phosphate-dibasic, calcium phosphate-tribasic, calcium sulfate, microcrystalline cellulose, silicified microcrystalline cellulose, cellulose powdered, dextrates, dextrins, dextrose excipients, fructose, kaolin, lactitol, lactose, mannitol, sorbitol, starch, starch pregelatinized, sucrose, sugar compressible, sugar confectioners and mixtures thereof.

Suitable disintegrants include one or more of starch, croscarmellose, crospovidone, sodium starch glycolate and the like. Suitable lubricants and glidants include one or more of colloidal anhydrous silica, stearic acid, magnesium stearate, calcium stearate, talc, hydrogenated caster oil, sucrose esters of fatty acid, microcrystalline wax, yellow beeswax, white beeswax and the like. Suitable plasticizers include one or more of polyethylene glycol, triethyl citrate, triacetin, diethyl phthalate, dibutyl sebacate and the like. Suitable stabilizers include one or more of antioxidants, buffers, acids and the like. Suitable coloring agents include any FDA approved colors for oral use.

The improved multiple unit systems described herein can be applied to most classes of drugs and most individual drugs. For example, two particular drugs that would benefit from an improved modified release multiple unit system are venlafaxine and glipizide. Venlafaxine is a potent inhibitor of neuronal serotonin and norepinephrine reuptake and is a weak inhibitor of dopamine reuptake. It is widely indicated for the treatment of depression and generalized anxiety disorder. The term “venlafaxine” as used herein includes venlafaxine base as well as any pharmaceutically acceptable salt thereof. Examples of pharmaceutically acceptable venlafaxine salts include venlafaxine hydrochloride. The venlafaxine layer weight may constitute from about 15% to about 75% of the total tablet weight.

Venlafaxine has been administered in the form of immediate release compressed tablets in doses ranging from 75 to 350 mg/day, in divided doses, two to three times a day. Such therapeutic dosing leads to wide fluctuations in the blood plasma levels of venlafaxine, with high concentrations at one extreme leading to severe side effects, such as nausea and/or vomiting shortly after administration, and less than therapeutic levels at the other extreme. Moreover, requiring frequent administration of the drug (e.g., two to three doses per day) is associated with patient non-compliance. Most of these problems associated with frequent dosing can be overcome by formulating controlled or extended release dosage forms of venlafaxine.

Venlafaxine hydrochloride is available as an extended release, once per day capsule which is marketed by Wyeth under the trade name Effexor® XR. This capsule appears to be described in U.S. Pat. No. 6,274,171, which discloses an extended release formulation of venlafaxine hydrochloride that includes spheroids of venlafaxine hydrochloride, microcrystalline cellulose, and optional hydroxypropyl methylcellulose coated with a mixture of ethylcellulose and hydroxypropyl methylcellulose. These film-coated spheroids are filled into capsules. However, these capsules suffer from a limitation that only a small number of coated beads or pellets can be put into a capsule of appropriate size that is convenient to swallow. Hence, there still exists a need for better controlled-release dosage forms of venlafaxine hydrochloride.

Glipizide is an oral blood glucose-lowering drug and is indicated as an adjunct to diet for the control of hyperglycemia and its associated symptoms in patients with non-insulin dependent diabetes mellitus. Glipizide stimulates secretion of insulin from the beta cells of pancreatic islet tissue and also exhibits extra-pancreatic action, including the ability to increase the number of insulin receptors. Chemically, glipizide is N-[2-[4-[[[(cyclohexylamino)carbonyl]amino]sulfonyl]phenyl]ethyl]-5-methylpyrazine carboxamide. Glipizide is a white, odorless powder with a pKa of 5.9, and is insoluble in both water and alcohol. These physicochemical properties of glipizide demand special techniques to formulate a dosage form that can be used to administer the drug at a controlled and predetermined rate.

Glipizide is available in the form of extended release oral tablets from Pfizer and is marketed under the trade name Glucotrol® XL. The extended release tablets are an osmotic drug delivery device that is based on push-pull technology. The delivery device includes a bi-layered core tablet that is coated with a semipermeable membrane having an orifice drilled on the coat for release of glipizide. The bilayered core tablet consists of a glipizide layer and a push layer of swellable polymers. When placed in dissolution media or gastrointestinal fluid, the device absorbs water through the semipermeable membrane, which leads to a swelling of the polymers in the push layer. This exerts a physical force on the drug layer forcing it out of the device through the orifice.

The glipizide layer of the pellets includes glipizide with or without other one or more of the pharmaceutically inert excipients described above. Optionally, this layer also may contain buffering agents. Buffers are used to maintain the pH of the glipizide layer and/or local environment surrounding the controlled release particles above to thereby aid in dissolution of glipizide in the dissolution media or gastrointestinal fluids. The buffering agents may be applied as an aqueous or non-aqueous solution or dispersion of drug in water/organic solvent, or mixtures thereof. Suitable buffering agents include one or more of dibasic sodium phosphate, sodium ascorbate, meglumine, sodium citrate trimethanolamine, sodium hydroxide, potassium hydroxide, calcium hydroxide, magnesium hydroxide, ammonia, tertiary sodium phosphate, diethanolamine, ethylenediamine, and L-lysine.

The inventors have developed improved multiple unit, controlled release tablets of venlafaxine that advantageously (1) can be administered in one half tablet or one half dosage and (2) can be prepared with a large amount of drug by compressing into a tablet of acceptable size that is easy to swallow. When administered, the controlled release tablet disintegrates rapidly into individual coated pellets of venlafaxine, which are dispersed into gastric fluid. Venlafaxine then is released in a controlled manner over a prolonged period of time from the individual coated pellets. Use of small controlled release coated pellets (i.e., units) decreases the chances of dose dumping and the performance of the units is also largely independent of gastric emptying time.

The improved multiple unit, controlled release tablet of venlafaxine can be prepared by processes known in the relevant art, e.g., comminuting, mixing, granulating, sizing, filling, molding, spraying, immersing, coating, compressing, etc.

In one of the embodiments, improved, multiple unit, controlled release tablets of venlafaxine can be prepared by coating inert pellets or cores with one or more venlafaxine layers which are further coated with a controlled release polymer layer. Optionally, the controlled release layer and/or venlafaxine layer may also be coated with a waxy layer to form the individual units. Further, these coated pellets or cores, or the units, may be blended with pharmaceutically acceptable excipients and compressed into suitably sized, multiple unit tablets.

Alternatively, the improved, multiple unit, controlled release tablets of venlafaxine can be prepared by coating inert pellets or cores with a single layer of venlafaxine and controlled release polymer. Optionally, the single layer of venlafaxine and polymer may be coated with a waxy layer to form the individual units. Further, these coated pellets or cores, or the units, may be blended with pharmaceutically acceptable excipients and compressed into suitably sized, multiple unit tablets.

The coating layers over the inert pellets or cores, or over the tablet, may be applied as a solution or dispersion of coating ingredients using any conventional technique known in the prior art, such as spray coating in a conventional coating pan or fluidized bed processor, dip coating, and the like. Alternatively, the layers over the inert pellet or core may be applied using a hot melt technique.

Optionally, the pellets or cores may be coated with one or more additional layers comprising film forming or sealing agents and/or pharmaceutically acceptable excipients between the above layers, over any of the layers, or over the inert pellet or core. The multiple unit tablets also may be further coated, if desired. Optionally, these additional coating layers over the tablet may comprise the active pharmaceutical ingredient (e.g., venlafaxine, glipizide) for immediate release. These layers may comprise film forming or sealing agents with or without other pharmaceutically acceptable excipients.

The improved, multiple unit systems described above are further illustrated by the following examples. Although these examples are illustrative of the techniques, compositions, and concepts described herein, they are not intended to be limiting.

### EXAMPLE 1

(A) Modified Release Multiple Units:

Example 1
(wt/tablet) mg

Inert Core

| | |
|---|---|
| Non pariel seeds | 65 |

Drug Layer

| | |
|---|---|
| Venlafaxine hydrochloride | 171 |
| | (equivalent to 150 mg |
| | of venlafaxine) |
| Magnesium stearate | 15 |
| Colloidal silica | 25 |
| Hydroxypropyl methylcellulose | 15 |
| Water | q.s |

Rate controlling layer

| | |
|---|---|
| Ethyl cellulose | 93.12 |
| Hydroxypropyl methylcellulose | 23.28 |
| Triacetin | 1% of total polymers |

Wax layer

| | |
|---|---|
| Polyethylene glycol 6000 | 30.55 |

Procedure:
1. Venlafaxine was dissolved in water and colloidal silica and then magnesium stearate and hydroxypropyl methylcellulose were added under stirring.
2. Non-pareil seeds were loaded in a Glatt Wurster column and coated with the drug dispersion of Step 1.
3. The drug coated pellets of Step 2 were coated with a mixture of ethyl cellulose and hydroxypropyl methylcellulose dissolved in a mixture of isopropyl alcohol and methylene chloride.
4. The coated pellets of Step 3 then were coated with a solution of PEG 6000 in methylene chloride.

(B) Compressed Tablet:

| | Example 1 |
|---|---|
| Ingredient | (wt/tablet) mg |
| Modified release multiple units of (A) | 438 |
| Silicified microcrystalline cellulose | 217 |
| PEG 4000 | 80 |
| Crospovidone | 90 |
| Magnesium Stearate | 5 |

Procedure: The modified release multiple units of (A) were mixed with other excipients and compressed to form tablets.

The compressed tablets prepared according to Example 1 had an acceptable hardness of about 7-13 Kp and disintegration times of about five minutes. Table 1 illustrates the comparative release patterns in vitro for modified release multiple units and tablets prepared according to Example 1.

TABLE 1
Comparative in vitro release patterns of modified release multiple
units and tablets using USP apparatus - II, at 50 rpm and pH 6.8.

| | |
|---|---|
| Time | Cumulative percentage release of venlafaxine |

| (Hours) | Modified release multiple units | Tablets |
|---|---|---|
| 1 | 14 | 17 |
| 2 | 32 | 33 |
| 4 | 59 | 57 |
| 6 | 72 | 69 |
| 8 | 82 | 79 |
| 12 | 94 | 91 |
| 16 | 100 | 97 |
| 20 | 100 | 100 |

As shown in Table 1, the compression of modified release multiple units into tablets did not alter the sustained release pattern of venlafaxine.

### EXAMPLE 2

(A) Modified Release Multiple Units:

Example 2
(wt/tablet) mg

Inert Core

| | |
|---|---|
| Non pariel seeds | 65 |

Drug Layer

| | |
|---|---|
| Venlafaxine hydrochloride | 171 |
| | (equivalent to 150 mg |
| | of venlafaxine) |
| Magnesium stearate | 13.5 |
| Colloidal silica | 19.7 |
| Hydroxypropyl methylcellulose | 13.5 |
| Water | q.s |

Rate controlling layer

| | |
|---|---|
| Ethyl cellulose | 93 |
| Hydroxypropyl methylcellulose | 24 |
| Triacetin | 1% of total polymers |

Wax layer

| | |
|---|---|
| Polyethylene glycol 6000 | 30 |

Procedure:
1. Venlafaxine was dissolved in water and colloidal silica and then magnesium stearate and hydroxypropyl methylcellulose were added under stirring.
2. Non-pareil seeds were loaded in a Glatt Wurster column and coated with the drug dispersion of Step 1.
3. The drug coated pellets of Step 2 were coated with a mixture of ethyl cellulose and hydroxypropyl methylcellulose that was dissolved in a mixture of isopropyl alcohol and methylene chloride.
4. The coated pellets of Step 3 then were coated with a solution of PEG 6000 in methylene chloride.

(B) Compressed Tablet:

| | Example 2 |
|---|---|
| Ingredient | (wt/tablet) mg |
| Modified release multiple units of (A) | 473 |
| Silicified microcrystalline cellulose | 288 |
| PEG 6000 | 71 |
| Crospovidone | 102 |
| Magnesium Stearate | 6 |

Procedure: The modified release multiple units of A were mixed with other excipients and compressed to form tablets.

The compressed tablets prepared according to Example 2 had an acceptable hardness of about 7-13 Kp and disintegration times of about five minutes. Table 2 illustrates the comparative release patterns in vitro for modified release multiple units and tablets prepared according to Example 2.

TABLE 2
Comparative in vitro release patterns of modified release multiple
units and tablets using USP apparatus - II, at 50 rpm and pH 6.8.

| | |
|---|---|
| Time | Cumulative percentage release of venlafaxine |

| (Hours) | Modified release multiple units | Tablets |
|---|---|---|
| 1 | 7 | 7 |
| 2 | 18 | 20 |
| 4 | 43 | 44 |
| 8 | 65 | 71 |
| 12 | 75 | 80 |

As shown in Table 2, the compression of modified release multiple units into tablets did not alter the sustained release pattern of venlafaxine.

### EXAMPLE 3

(A) Modified Release Multiple Units:

Example 3
(wt/tablet) mg

Inert Core

| | |
|---|---|
| Celpheres | 148 |

Drug Layer

| | |
|---|---|
| Glipizide | 10 |
| Polyethylene glycol | 4.7 |
| Hydroxypropyl methylcellulose | 1.7 |
| Polyvinyl pyrrolidone | 3.0 |
| Tween 80 | 0.5 |
| Lactose | 3.0 |

Rate controlling layer

| | |
|---|---|
| Ethyl cellulose | 8 |
| Hydroxypropyl methylcellulose | 4 |
| Triacetin | 1.3 |
| Talc | 0.4 |

Wax layer

| | |
|---|---|
| Polyethylene glycol 6000 | 13.9 |

Procedure:
1. Polyethylene glycol, hydroxypropyl methylcellulose, polyvinyl pyrrolidone, Tween and lactose were dissolved in water and glipizide then was dispersed in the solution.
2. Celpheres were loaded in a Glatt Wurster column and coated with the drug dispersion of Step 1.
3. A solution of ethyl cellulose, hydroxypropyl methylcellulose and triacetin was prepared in a mixture of methylene chloride and isopropyl alcohol into which talc was dispersed.
4. The drug loaded pellets of Step 2 then were coated with the dispersion of Step 3 using a Glatt Wurster column.
5. The coated pellets of Step 4 then were coated with a solution of PEG 6000 in mixture of isopropyl alcohol and methylene chloride.

(B) Compressed Tablet:

| | Example 3 |
|---|---|
| Ingredient | (wt/tablet) mg |
| Modified release multiple units of (A) | 197.4 |
| Silicified microcrystalline cellulose | 122.4 |
| PEG 6000 | 29.6 |
| Crospovidone | 43.4 |
| Magnesium Stearate | 2.0 |

Procedure: The modified release multiple units of (A) were mixed with other excipients and compressed to form tablet

The compressed tablets prepared according to Example 3 had an acceptable hardness of about 8-10 Kp and disintegration time of about three minutes. Tables 3a and 3b illustrate the comparative release patterns in vitro for modified release multiple units and tablets, respectively, prepared according to Example 3.

TABLE 3a
In vitro release pattern of modified release multiple
units using USP apparatus - II, at 50 rpm and pH 7.5

| | Cumulative percentage release of glipizide |
|---|---|
| Time (Hours) | from modified release multiple units |
| 1 | 6 |
| 2 | 13 |
| 4 | 23 |
| 8 | 45 |
| 12 | 62 |
| 16 | 78 |
| 20 | 94 |
| 24 | 102 |

TABLE 3b
In vitro release pattern of tablets using
USP apparatus - II, at 50 rpm and pH 7.5

| | Cumulative percentage release of glipizide |
|---|---|
| Time (Hours) | from tablets |
| 0.3 | 3 |
| 2.3 | 18 |
| 6.3 | 44 |
| 10.3 | 65 |
| 14.3 | 83 |
| 18.3 | 100 |
| 22.3 | 107 |

As shown in Tables 3a and 3b above, the compression of modified release multiple units into tablets did not alter the sustained release pattern of glipizide.

The above examples illustrate that the techniques, compositions, and concepts described herein can provide modified release multiple unit systems that can withstand the mechanical stresses of tablet formation without affecting the desired release characteristics.

### EXAMPLES 4-7

Additional formulations of controlled release tablets of venlafaxine prepared according to the compositions of Examples 4-7 are provided in Tables 4 and 5

TABLE 4
Composition of coated pellets

| | | | |
|---|---|---|---|
| Example | Example | Example | Example |
| 4 (wt/ | 5 (wt/ | 6 (wt/ | 7 (wt/ |
| tablet) | tablet) | tablet) | tablet) |
| mg | mg | mg | mg |

Inert pellets

| | | | | |
|---|---|---|---|---|
| Non pariel seeds | 65 | 65 | 65 | 65 |

Venlafaxine layer

| | | | | |
|---|---|---|---|---|
| Venlafaxine | 171 | 171 | 171 | 171 |
| hydrochloride | | | | |
| Magnesium stearate | 13.5 | 13.5 | 13.55 | 13.55 |
| Colloidal silica | 19.7 | 19.7 | 19.70 | 19.70 |
| Hydroxypropyl methyl | 13.5 | 13.5 | 13.55 | 13.55 |
| cellulose | | | | |
| Water | q.s | q.s | q.s | q.s |

Controlled release polymer layer

| | | | | |
|---|---|---|---|---|
| Ethyl cellulose | 81.42 | 91.61 | 101.77 | 110.84 |
| Hydroxypropyl | 20.35 | 22.89 | 25.44 | 27.68 |
| methylcellulose | | | | |
| Triacetin | 1.01 | 1.14 | 1.27 | 1.38 |

Waxy layer

| | | | | |
|---|---|---|---|---|
| Polyethylene glycol 6000 | 28.8 | 30 | 30.72 | 33.27 |

Procedure:
1. A solution of venlafaxine hydrochloride was prepared in water. Colloidal silica, magnesium stearate and hydroxypropyl methylcellulose were added to the solution under stirring to form a uniform dispersion.
2. Non pareil seeds were loaded in a Glatt Wurster column and coated with the drug dispersion of Step 1.
3. The venlafaxine coated pellets of Step 2 then were coated with a solution of ethyl cellulose and hydroxypropyl methylcellulose that was dissolved in a mixture of isopropyl alcohol and methylene chloride.

4. The coated pellets of Step 3 then were coated with a solution of Polyethylene glycol 6000 in isopropyl alcohol and methylene chloride.

TABLE 5
Composition of controlled release venlafaxine tablets

| | Example | Example | Example | Example |
|---|---|---|---|---|
| | 4 (wt/ | 5 (wt/ | 6 (wt/ | 7 (wt/ |
| | tablet) | tablet) | tablet) | tablet) |
| Ingredient | mg | mg | mg | mg |
| Coated Pellets | 459 | 473 | 450 | 465 |
| Silicified | 288 | 288 | 276 | 285 |
| microcrystalline | | | | |
| cellulose | | | | |
| Polyethylene glycol | 70 | 71 | 85 | 89 |
| 6000 | | | | |
| Crospovidone | 102 | 102 | 98 | 100 |
| Magnesium | 6 | 6 | 6 | 6 |
| Stearate | | | | |

Procedure:

The coated pellets were blended with silicified microcrystalline cellulose, polyethylene glycol 6000, and crospovidone; lubricated with magnesium stearate; and compressed into suitably sized tablets.

In Vitro Dissolution Study

The in vitro release of venlafaxine hydrochloride from controlled release tablets made according to the compositions of Examples 4-7 was studied in 900 ml of phosphate buffer (pH-6.8) using USP apparatus-II, at 50 rpm. The results of this testing are listed in Table 6.

TABLE 6
In vitro release of venlafaxine hydrochloride
from controlled release tablets

| | |
|---|---|
| | Cumulative percentage (%) release |
| Time | of venlafaxine from tablets |

| (Hours) | Example 4 | Example 5 | Example 6 | Example 7 |
|---|---|---|---|---|
| 1 | 7 | 7 | 4 | 3 |
| 2 | 24 | 20 | 12 | 11 |
| 4 | 51 | 44 | 34 | 30 |
| 8 | 79 | 71 | 57 | 53 |
| 12 | 91 | 80 | 68 | 64 |
| 14 | 95 | 84 | 72 | 68 |
| 16 | 98 | 88 | 75 | 71 |
| 18 | 101 | 90 | 76 | 74 |
| 20 | 102 | 91 | 79 | 76 |
| 24 | 102 | 95 | 82 | 80 |

In Vivo Bioavailability Study

The in vivo performance of venlafaxine hydrochloride tablets prepared as per the composition of Examples 4 and 5 were evaluated with respect to the Effexor® XR 150 mg capsules in 11 healthy male volunteers under fasting condition. The study protocol followed was open randomized 3 treatment, 3 period, 6 sequence cross over study with a wash out period of at least 5 days. Blood samples were collected at appropriate time intervals over a period of 48 hours and venlafaxine content analyzed using a validated inhouse LCMS-MS method. Pharmacokinetic parameters Cₘₐₓ (Maximum plasma concentration), Tₘₐₓ (Time to attain maximum plasma concentration), AUC₀₋ₜ (Area under the plasma concentration vs time curve from 0 hours to the time of last sample collected) and AUC_{0-α} (Area under the plasma concentration vs. time curve from 0 hours to infinity) were calculated from the data obtained. The results of the study are given in Table 7.

TABLE 7
Comparative pharmacokinetic data

| | Tₘₐₓ | Cₘₐₓ | AUC₀₋ₜ | AUC_{0-∝} |
|---|---|---|---|---|
| Pharmacokinetic parameter | (h) | μg/ml | (μg/ml) (h) | (μg/ml) (h) |
| Tablets of Example 4 | 4.85 | 114.31 | 1633.51 | 1795.72 |
| Tablets of Example 5 | 5.091 | 130.56 | 1813.84 | 2006.79 |
| Effexor ® XR capsules | 6.45 | 99.92 | 1719.49 | 2406.27 |

The controlled release tablets produced demonstrated comparable extent of absorption when compared to the reference Effexor® XR. It is within the skill of one ordinary skill in the art to develop a product with matching Cₘₐₓ and AUC₀₋ₜ with respect to the reference product. The controlled release tablets can provide therapeutic blood concentrations of venlafaxine over a period of at least twenty four hours.

Examples 8 and 9, described below, provide additional examples of controlled release, multiple unit formulations of glipizide that deliver glipizide over twenty four hours. In contrast to Example 3 of a glipizide formulation having a waxy layer, these glipizide examples have the rate controlling polymer layer but not the waxy layer.

### EXAMPLE 8

Controlled Release Multiple Units:

Example 8
(wt/tablet) mg

Inert Core

| | |
|---|---|
| Celpheres | 148 |

Drug Layer

| | |
|---|---|
| Glipizide | 10 |
| Polyethylene glycol | 4.7 |
| Hydroxypropyl methylcellulose | 1.7 |
| Polyvinyl pyrrolidone | 3.0 |
| Tween 80 | 0.5 |
| Lactose | 3.0 |

Rate controlling layer

| | |
|---|---|
| Ethyl cellulose | 10 |
| Hydroxypropyl methylcellulose | 5 |
| Triacetin | 1.7 |
| Talc | 0.5 |

Procedure:
1. Polyethylene glycol, hydroxypropyl methylcellulose, polyvinyl pyrrolidone, Tween and lactose were dissolved in water and glipizide then was dispersed in the solution.
2. Celpheres were loaded in a Glatt Wurster column and coated with the drug dispersion of Step 1.
3. A solution of ethyl cellulose, hydroxypropyl methylcellulose and triacetin was prepared in a mixture of methylene chloride and isopropyl alcohol into which talc was dispersed.
4. The drug loaded pellets of Step 2 then were coated with the dispersion of Step 3 using a Glatt Wurster column to prepare controlled release multiple units.

Table 8 illustrates the comparative release patterns in vitro for the controlled release multiple units prepared according to example 8.

TABLE 8
In vitro release pattern of controlled release multiple
units using USP apparatus - II, at 50 rpm and pH 7.5

| | Cumulative percentage release of glipizide |
|---|---|
| Time (Hours) | from controlled release multiple units |
| 1 | 10 |
| 2 | 18 |
| 4 | 29 |
| 8 | 46 |
| 12 | 62 |
| 16 | 74 |
| 20 | 89 |
| 24 | 98 |

### EXAMPLE 9

Controlled Release Multiple Units:

Example 9
(wt/tablet) mg

Inert Core

| | |
|---|---|
| Celpheres | 148 |

Drug Layer

| | |
|---|---|
| Glipizide | 10.0 |
| Polyethylene glycol | 4.7 |
| Hydroxypropyl methylcellulose | 1.7 |
| Polyvinyl pyrrolidone | 3.0 |
| Tween 80 | 0.5 |
| Lactose | 3.0 |

Rate controlling layer

| | |
|---|---|
| Ethyl cellulose | 4.6 |
| Hydroxypropyl methylcellulose | 2.9 |
| Triacetin | 0.8 |
| Talc | 0.3 |

Procedure:
1. Polyethylene glycol, hydroxypropyl methylcellulose, polyvinyl pyrrolidone, lactose and Tween were dissolved in water and glipizide then was dispersed in the solution.
2. Celpheres were loaded in a Glatt Wurster column and coated with the drug dispersion of Step 1.
3. A solution of ethyl cellulose, hydroxypropyl methylcellulose and triacetin was prepared in a mixture of methylene chloride and isopropyl alcohol into which talc was dispersed.
4. The drug loaded pellets of Step 2 then were coated with the dispersion of Step 3 using a Glatt Wurster column to prepare controlled release multiple units.

Table 9 illustrates the comparative release patterns in vitro for controlled release multiple units prepared according to Example 9.

TABLE 9
In vitro release pattern for controlled release multiple
units using USP apparatus - II, at 50 rpm and pH 7.5

| | Cumulative percentage release of glipizide |
|---|---|
| Time (Hours) | from controlled release multiple units |
| 1 | 26 |
| 2 | 37 |
| 4 | 55 |
| 8 | 74 |
| 12 | 86 |
| 16 | 93 |
| 20 | 97 |
| 24 | 98 |

Tables 8 and 9 indicate that controlled release, multiple unit systems of glipizide can be prepared that can provide therapeutic blood concentrations of glipizide over a period of at least twenty four hours.

While several particular forms of the inventions have been described, it will be apparent that various modifications and combinations of the inventions detailed in the text can be made without departing from the spirit and scope of the inventions. For example, the waxy layer can, for example, affect the release of the units, or a mixture of a waxy material and a functional material, such as an active pharmaceutical ingredient or a functional pharmaceutical excipient. The mixture of waxy material and active pharmaceutical ingredients may provide an immediate release of the active pharmaceutical ingredient on the mixture. The waxy layer can be designed based on, for example, thickness or material to impart rate controlling properties to the units or pellets. The improved multiple unit systems also generally are intended for application to any active pharmaceutical ingredient and provide advantages to those that are primarily formulated as a capsule and/or are problematic to prepare as a tablet. Moreover, the multiple unit systems can be prepared as a tablet, capsule, or sachet that includes a core and a coating of a waxy material. The core can consist of one or more active pharmaceutical ingredients and those pharmaceutically acceptable excipients necessary to form the core. The coating of waxy material allows the coated cores (i.e., units) to be compress as a tablet or filled into a capsule or sachet. In this manner, the dosage form can be immediate release. By adding a rate controlling polymer to the core, the dosage form can be an extended release. The dosage form also can be made from a mixture of immediate release and extended release units to provide immediate and extended release of the one or more active pharmaceutical ingredients.

Pharmaceutically acceptable salts of venlafaxine and glipizide may be used in the dosage forms, tablets, and capsules described herein. Pharmaceutically acceptable salts of venlafaxine and glipizide include mineral acid salts such as hydrochloride, hydroiodide, hydroflouride, sulphate, etc.; organic acid salts such as citrate, maleate, tartarate, etc.; and organosulphonic acid salts such as mesylate, besylate, tosylate, etc.

The improved multiple unit systems can be used to deliver combination drug products, such as combinations of atorvastatin and amlodipine, metformin and glipizide, simvastatin and ramipril, simvastatin and amlodipine, metformin XL and glipizide XL, ramipril and atorvastatin, ramipril and amlodipine, metformin XL and glimiperide, fosinopril and amlodipine. These combination drug products can be produced by separately forming individual units of each active pharmaceutical ingredient and then combining them into tablets, capsules, or sachets in a subsequent production step. In this manner, each of the active pharmaceutical ingredients can be fabricated to separately optimize the release of that active ingredient and then the final dosage form can be produced that has the desired ratio of each of the active ingredients. One or both of each of the active ingredients can be formed as units of one or more of an immediate release, a controlled release, a modified release, a delayed release, or an extended release form.

Further, it is contemplated that any single feature or any combination of optional features of the inventive variations described herein may be specifically excluded from the claimed inventions and be so described as a negative limitation. Accordingly, it is not intended that the inventions be limited, except as by the appended claims.

1. A multiple unit dosage form comprising multiple units, each unit comprising: at least one core having an outer surface;
a first coating layer surrounding at least a portion of the outer surface of the core and having an outer surface, the coating layer including one or both of one or more active pharmaceutical ingredients and one or more rate controlling polymers; and
an outer layer, the outer layer comprising a material that is one or both of elastic and compressible.

2. The multiple unit dosage form of claim 1, wherein the core includes the one or more rate controlling polymers.

3. The multiple unit dosage form of claim 1, wherein the core includes the one or more active pharmaceutical ingredients.

4. (canceled)

5. (canceled)

6. (canceled)

7. The multiple unit dosage form of claim 1, wherein the rate controlling polymer comprises one or more of cellulosic polymers, methacrylic acid polymers, and waxes.

8. (canceled)

9. The multiple unit dosage form of claim 1, wherein the one or more active pharmaceutical ingredients comprises one or more of antidepressants, antidiabetics, antiulcers, analgesics, antihypertensives, antibiotics, antipsychotics, antineoplastics, antimuscarinics, diuretics, antimigraine agents, antivirals, anti-inflammatory agents, sedatives, antihistaminics, antiparasitic agents, antiepileptics and lipid lowering agents.

10. The multiple unit dosage form of claim 1, wherein the one or more active pharmaceutical ingredients comprise one or more of enalapril, captopril, benazepril, lisinopril, ranitidine, famotidine, ranitidine bismuth citrate, diltiazem, propranolol, verapamil, nifedipine, acyclovir, ciprofloxacin, simvastatin, atorvastatin, lovastatin, venlafaxine, citalopram, paroxetine, selegiline, midazolam, fluoxetine, acarbose, buspirone, nimesulide, captopril, nabumetone, glimepiride, glipizide, etodolac, nefazodone and their pharmaceutically acceptable salts.

11. The multiple unit dosage form of claim 1, wherein the one or more active pharmaceutical ingredients comprises one or both of glipizide and venlafaxine or their salts.

12. The multiple unit dosage form of claim 1, wherein the core includes the rate controlling polymer and the active pharmaceutical ingredient.

13. The multiple unit dosage form of claim 1, wherein the first coating layer further includes the active pharmaceutical ingredient.

14. The multiple unit dosage form of claim 1, wherein the first coating layer includes the one or more active pharmaceutical ingredients.

15. The multiple unit dosage form of claim 1, further comprising one or more additional layers, wherein the additional layers are positioned between (a) one or more of the core and the first coating layer and (b) surrounding at least a portion of the first coating layer,
wherein the one or more additional layers comprise one or more of a seal coat, a film forming layer, a rate controlling polymer, and an active pharmaceutical ingredient.

16. (canceled)

17. (canceled)

18. The multiple unit dosage form of claim 1, wherein the material in the outer layer comprises one or more wax materials.

19. (canceled)

20. (canceled)

21. (canceled)

22. (canceled)

23. (canceled)

24. (canceled)

25. (canceled)

26. The multiple unit dosage form of claim 1, wherein the active pharmaceutical ingredient comprises glipizide and is in one or both of the core and the first coating layer.

27. The multiple unit dosage form of claim 26, further comprising a buffering agent with the glipizide in one or both of the core and the first coating layer.

28. (canceled)

29. The multiple unit dosage form of claim 1, wherein one or more of the core and the first coating layer includes one or more pharmaceutically acceptable excipients.

30. (canceled)

31. (canceled)

32. (canceled)

33. (canceled)

34. (canceled)

35. (canceled)

36. (canceled)

37. The multiple unit dosage form of claim 1, wherein the dosage form comprises a tablet.

38. The multiple unit dosage form of claim 37, wherein the tablet further includes one or more pharmaceutically acceptable excipients around the individual units.

39. The multiple unit dosage form of claim 1, wherein the dosage form comprises a capsule.

40. The multiple unit dosage form of claim 1, wherein the active pharmaceutical ingredients comprise one or more of atorvastatin and amlodipine, metformin and glipizide, simvastatin and ramipril, simvastatin and amlodipine, metformin XL and glipizide XL, ramipril and atorvastatin, ramipril and amlodipine, metformin XL and glimiperide, fosinopril and amlodipine.

41. A process for the preparation of a multiple unit dosage form, the process comprising:
providing at least one core having an outer surface;
forming a coated core by applying one or more coating layers to the core such that the one or more coating layers surround at least a portion of the outer surface of the core or the coating layers;
forming an individual unit by applying a waxy material to the coated core to form a wax layer;
combining one or more units to form a multiple unit dosage form,
wherein one or both of the core and the coating layers includes one or more rate controlling polymers and active pharmaceutical ingredients.

42. The process of claim 41, further comprising applying one or both of a seal layer or a film forming layer between the core and the coating layer, between the one or more coating layers, and between the one or more coating layers and the wax layer.

43. (canceled)

44. (canceled)

45. (canceled)

46. (canceled)

47. (canceled)

48. (canceled)

49. (canceled)

50. (canceled)

51. (canceled)

52. The process of claim 41, wherein the core comprises one or more pharmaceutically acceptable excipients.

53. The process of claim 41, wherein the core comprises one or more active pharmaceutical ingredients.

54. The process of claim 41, wherein the one or more active pharmaceutical ingredients comprises one or more of antidepressants, antidiabetics, antiulcers, analgesics, antihypertensives, antibiotics, antipsychotics, antineoplastics, antimuscarinics, diuretics, antimigraine agents, antivirals, anti-inflammatory agents, sedatives, antihistaminics, antiparasitic agents, antiepileptics and lipid lowering agents.

55. The process of claim 41, wherein the one or more active pharmaceutical ingredients comprise one or more of enalapril, captopril, benazepril, lisinopril, ranitidine, famotidine, ranitidine bismuth citrate, diltiazem, propranolol, verapamil, nifedipine, acyclovir, ciprofloxacin, simvastatin, atorvastatin, lovastatin, venlafaxine, citalopram, paroxetine, selegiline, midazolam, fluoxetine, acarbose, buspirone, nimesulide, captopril, nabumetone, glimepiride, glipizide, etodolac, nefazodone and their pharmaceutically acceptable salts.

56. (canceled)

57. (canceled)

58. (canceled)

59. (canceled)

60. The process of claim 41, wherein the units are prepared by coating the cores with active pharmaceutical ingredients and rate controlling polymers.

61. The process of claim 41, wherein the units are prepared by coating cores with a first layer comprising an active pharmaceutical ingredient and a second outer layer comprising a rate controlling polymer.

62. (canceled)

63. The process of claim 41, wherein the rate controlling polymer comprises one or more of cellulosic polymers, methacrylic acid polymers, waxes, ethylcellulose, hydroxypropyl methylcellulose, hydroxypropyl cellulose, methylcellulose, carboxymethylcellulose, hydroxymethylcellulose, hydroxyethylcellulose, hydroxypropylmethyl phthalate, cellulose acetate phthalate, and cellulose acetate trimellitate.

64. The process of claim 41, wherein the active pharmaceutical ingredient comprises venlafaxine.

65. The process of claim 41, wherein the active pharmaceutical ingredient comprises glipizide.

66. (canceled)

67. (canceled)

68. A method for preparing a modified release multiple unit dosage form, the method comprising:
providing a core having a coating, wherein one or both of the core and the coating include one or more of rate controlling polymers and active pharmaceutical ingredients;
forming individual units by coating the coated core with a coating material that is one or both of compressible and elastic; and
forming the dosage form by combining one or more individual units.

69. (canceled)

70. (canceled)

71. (canceled)

72. (canceled)

73. A method of treating a medical condition, the method comprising administering a multiple unit dosage form for oral ingestion, each unit comprising a core, one or more layers surrounding the core, and an outer layer, wherein
the core comprises one or more of a pharmaceutically acceptable excipients, an active pharmaceutical ingredient, and a rate controlling polymer,
the one or more layers comprises one or more of a pharmaceutically acceptable excipient, an active pharmaceutical ingredient, a rate controlling polymer, a sealing layer, and a film forming layer, and
the outer layer comprises a material that is one or both of compressible and elastic to partially or completely absorb a force exerted in forming the multiple unit dosage form by combining the units.

74. The method of claim 73, wherein the material of the outer layer comprises a waxy material.

75. (canceled)

76. (canceled)

77. (canceled)

78. A multiple unit dosage form comprising multiple units, each unit comprising:
at least one core having an outer surface and comprising one or more one active pharmaceutical ingredients; and
a coating layer surrounding at least a portion of the outer surface of the core, having an outer surface and comprising a waxy material.

79. The multiple unit dosage form of claim 78, wherein the waxy material comprises one or more polyethylene glycols of different molecular weights.

80. (canceled)

81. (canceled)

82. A combination drug, multiple unit dosage form comprising:
first units; and
second units,
each first unit comprising at least one core having an outer surface, a first coating layer surrounding at least a portion of the outer surface of the core and having an outer surface, and an outer layer surrounding at least a portion of an outer surface of the first coating layer, the first coating layer including a first active pharmaceutical ingredient,
each second unit comprising at least one core having an outer surface, a first coating layer surrounding at least a portion of the outer surface of the core and having an outer surface, and an outer layer surrounding at least a portion of an outer surface of the first coating layer, the first coating layer including a second active pharmaceutical ingredient,
wherein one or both of the cores and the coating layers comprise a rate controlling polymer, and
one or both of the outer layers comprise a waxy material.

83. The combination drug, multiple unit dosage form of claim 82, wherein the waxy material comprises one or more polyethylene glycols.

84. (canceled)

84. (canceled)

85. A multiple unit dosage form comprising multiple units, each unit comprising:
at least one core having an outer surface;
a first coating layer surrounding at least a portion of the outer surface of the core and having an outer surface, the coating layer including glipizide or its pharmaceutically acceptable salt and optionally one or more rate controlling polymers.

86. The multiple unit dosage form of claim 85, wherein the pharmaceutically acceptable salt comprises one or more of mineral acid salts, organic acid salts, and organosulphonic acid salts.

87. (canceled)

88. (canceled)

89. (canceled)

90. (canceled)

91. The multiple unit dosage form of claim 85, wherein the core includes rate controlling polymer and glipizide.

92. The multiple unit dosage form of claim 85, further comprising one or more additional layers, wherein the additional layers are positioned between (a) one or more of the core and the first coating layer and (b) surrounding at least a portion of the first coating layer,
wherein the one or more additional layers comprise one or more of a seal coat, a film forming layer, a rate controlling polymer, and an active pharmaceutical ingredient.

93. (canceled)

94. The multiple unit dosage form of claim 85, further comprising an outer layer, the outer layer comprising a material that is one or both of elastic and compressible.

95. The multiple unit dosage form of claim 94, wherein the material in the outer layer comprises one or more wax materials.

96. (canceled)

97. The multiple unit dosage form of claim 85, further comprising a buffering agent with the glipizide in the first coating layer.

98. (canceled)

99. (canceled)

100. (canceled)

101. A modified release multiple unit system comprising units of glipizide, wherein the units comprise:
an inert core;
a drug layer surrounding the inert core, the drug layer comprising glipizide; and
a rate controlling polymer layer surrounding the drug layer.

102. (canceled)

103. (canceled)

104. A modified release multiple unit system comprising units of glipizide wherein the units comprise:
an inert core;
a drug layer surrounding the inert core;
a rate controlling polymer layer surrounding the drug layer; and
a waxy layer surrounding the drug layer.

105. The modified release multiple unit system of claim 104, wherein the units can be compressed into tablet, or filled into a capsule or a sachet; without affecting the desired release characteristics of drug.

106. (canceled)

107. (canceled)

108. A modified release multiple unit system comprising units of venlafaxine, wherein the units comprise:
an inert core;
a drug layer surrounding the inert core; and
a rate controlling polymer layer surrounding the drug layer.

109. (canceled)

110. A modified release multiple unit system comprising units of venlafaxine wherein the units comprise:
an inert core;
a drug layer surrounding the inert core;
a rate controlling polymer layer surrounding the drug layer; and
a waxy layer surrounding the rate controlling polymer layer.

111. The modified release multiple unit system of claim 110, wherein the units can be compressed into tablet without affecting the desired release characteristics of drug.

112. A modified release multiple unit system comprising units of a drug wherein the units comprise:
an inert core;
a drug layer surrounding the inert core;
a rate controlling polymer layer surrounding the drug layer; and
a waxy layer surrounding the rate controlling polymer layer.

113. The modified release multiple unit system of claim 112, wherein the units can be compressed into tablet, or filled in capsule or sachet; without affecting the desired release characteristics of drug.

114. A process for the preparation of a modified release multiple unit system of a drug, the process comprising the steps of:
coating inert pellets with a drug and rate controlling polymer layer;
coating with a waxy layer;
optionally blending with pharmaceutically acceptable excipients;
compressing into a tablet, or filling into a capsule or a sachet of suitable size.

115. A process for the preparation of a modified release multiple unit system of drug, the process comprising the steps of:
coating inert pellets with a drug and rate controlling polymer layer;
coating with a waxy layer;
optionally blending with pharmaceutically acceptable excipients;
compressing into tablet of suitable size.

116. The process of claim 115, wherein the drug comprises venlafaxine or a pharmaceutically acceptable salt.

117. A process for the preparation of modified release multiple unit system of drug comprising the steps of:
coating drug containing cores with a rate controlling polymer layer;
coating the rate controlling polymer layer with a waxy layer;
optionally blending with pharmaceutically acceptable excipients; and
compressing into tablet, or filling into capsule or sachet of suitable size
